(19) United States
(12) Patent Application Publication
(10) Pub. No.: US 2026/0201434 A1
(43) Pub. Date: Jul. 16, 2026

(54) METHOD AND SYSTEM FOR PRODUCING PURIFIED GALACTO-OLIGOSACCHARIDE DERIVED FROM ACID WHEY OR PASTEURIZED WHEY

(71) Applicant: Chobani LLC, Norwich, NY (US)

(72) Inventors: Byron Toledo, Twin Falls, ID (US); Guru Yeshwanth Subbiah Prabhakaran, Highland Park, NJ (US); Marc Abjean, Twin Falls, ID (US); Archit Shivanshu Mehta, Twin Falls, ID (US)

(21) Appl. No.: 19/022,314

(22) Filed: Jan. 15, 2025

(51) Int. Cl. C12P 19/12; C07H 1/08; C12N 9/04; C12N 9/08; C12N 9/38

(52) U.S. Cl. CPC C12P 19/12; C07H 1/08; C12N 9/0006; C12N 9/0065; C12N 9/2471; C12Y 101/03004; C12Y 111/01006; C12Y 302/01023

(57) ABSTRACT

A method for manufacturing a dairy-derived galacto-oligosaccharide product. In a first aspect of the invention, the method comprises pasteurization, ultrafiltration, nanofiltration, electrodialysis, ion exchange chromatography, evaporation, pH adjustment, enzyme reaction to form galacto-oligosaccharide, enzyme deactivation, additional ultrafiltration, additional ion exchange chromatography, additional evaporation, fractionation chromatography, mixing a portion of the second evaporation concentrate with the fractionation portion, polishing ion exchange chromatography, and yet another evaporation. A second aspect of the invention includes all above steps and further includes an additional evaporation step after the fractionation chromatography step. A third aspect of the invention includes all above steps and, between the mixing step and polishing ion exchange chromatography step, further includes a cooling step, additional enzyme reaction step, additional enzyme deactivation step, additional ultrafiltration, and an additional ion exchange chromatography step. These aspects form differing grades of galacto-oligosaccharide products having different percentages of galacto-oligosaccharide, galactose, and glucose.

### TECHNICAL FIELD

This application relates to food products and methods and, in particular, relates to a dairy-derived galacto-oligosaccharide product and a method of making a dairy-derived galacto-oligosaccharide product.

### BACKGROUND

In the manufacture of food products, galacto-oligosaccharides, complex mixtures of various sugars, are produced by transgalactosylation from lactose using β-galactosidase and are of great interest for food and feed applications because of their prebiotic properties. Most galacto-oligosaccharide preparations currently available in the market contain a significant amount of monosaccharides and lactose.

There is a modern trend to supply nutritious, prebiotic food with little to no sugar content. Accordingly, it would be desirable to produce food products from galacto-oligosaccharides with reduced concentrations of glucose and lactose. Of course, maintaining previously-made improvements in flavoring, color, and desirability of such food products is also an objective when making alternatives with lower sugar content.

### SUMMARY

To address these and other technical problems within the conventional art, a method for manufacturing a dairy-derived galacto-oligosaccharide product is provided. In a first aspect of the invention, the method includes: subjecting a pasteurized whey mixture having an original concentration of lactose to a first ultrafiltration step to form a first ultrafiltration retentate and a first ultrafiltration permeate; subjecting the first ultrafiltration permeate to a first separation step to form a first separation permeate and a first separation retentate, wherein the first separation step consists of either a nanofiltration separation or a reverse osmosis separation; subjecting the first separation retentate to a first evaporation step to form a first evaporation concentrate and a first evaporation condensate; adjusting a pH of the first evaporation concentrate to a value greater than or equal to 3 and less than or equal to 9 to form a pH-adjusted first evaporation concentrate; reacting, to form a galacto-oligosaccharide, at least one enzyme with the pH-adjusted first evaporation concentrate by adding the at least one enzyme to the pH-adjusted first evaporation concentrate to form a first reaction product comprising the galacto-oligosaccharide; subjecting the first reaction product to a second ultrafiltration step to form a second ultrafiltration retentate and a second ultrafiltration permeate, the second ultrafiltration permeate being poorer in a protein concentration than the second ultrafiltration retentate; subjecting the second ultrafiltration permeate to a second ion exchange chromatography step comprising reducing one or more mineral concentrations in the second ultrafiltration permeate to form a second mineral-scarce mixture; subjecting the second mineral-scarce mixture to a second evaporation step to form a second evaporation concentrate and a second evaporation condensate; separating a portion of the second evaporation concentrate to form a second evaporation concentrate bypass mixture and a second evaporation concentrate line mixture; subjecting the second evaporation concentrate line mixture to a fractionation chromatography step to form a first fractionation portion and a second fractionation portion, wherein the first fractionation portion comprises the galacto-oligosaccharide; mixing the first fractionation portion and the second evaporation concentrate bypass mixture to form a commodity mixture; subjecting the commodity mixture to a polishing ion exchange chromatography step to form a polished commodity mixture; and subjecting the polished commodity mixture to a third evaporation step to form a third evaporation condensate and a third evaporation concentrate, the third evaporation concentrate comprising the dairy-derived galacto-oligosaccharide product, the dairy-derived galacto-oligosaccharide product comprising lactose in a concentration less than the original concentration of lactose.

In one embodiment of the first aspect of the invention, the method further comprises, prior to subjecting the pasteurized whey mixture to the first ultrafiltration step, pasteurizing a clarified whey mixture comprising a dairy-derived acid whey and the original concentration of lactose, to form the pasteurized whey mixture. In a further embodiment thereof, pasteurizing the clarified whey mixture comprises subjecting the clarified whey mixture to a high-temperature, short-time pasteurization process, wherein the high-temperature, short-time pasteurization process is conducted at a temperature greater than or equal to 161° F. and for a duration greater than or equal to 15 seconds.

In one embodiment of the first aspect of the invention, the first ultrafiltration step comprises filtering the pasteurized whey mixture through an ultrafilter having an average pore size of between 5,000 Daltons and 10,000 Daltons.

In one embodiment of the first aspect of the invention, the first separation step consists of a nanofiltration separation, wherein the nanofiltration separation comprises filtering the ultrafiltration permeate through a nanofilter having an average pore size of between 150 Daltons and 300 Daltons.

In one embodiment of the first aspect of the invention, the method further comprises, prior to the first evaporation step, subjecting the first separation retentate to a first electrodialysis step comprising reducing one or more mineral concentrations in the first separation retentate. In one such embodiment, the one or more mineral concentrations reduced in the first electrodialysis step comprise a mineral selected from a group consisting of calcium, magnesium, phosphorus, potassium, sodium, and combinations thereof.

In one embodiment of the first aspect of the invention, the method further comprises, prior to the first evaporation step, subjecting the first separation retentate to a first ion exchange chromatography step comprising reducing one or more mineral concentrations in the first separation retentate. In one such embodiment, the one or more mineral concentrations reduced in the first ion exchange chromatography step comprise a mineral selected from a group consisting of calcium, magnesium, phosphorus, potassium, sodium, and combinations thereof.

In one embodiment of the first aspect of the invention, adjusting the pH of the first evaporation concentrate comprises adjusting the pH to a value in a pH range selected from the group consisting of greater than or equal to 3 and less than or equal to 7, alternatively greater than or equal to 5 and less than or equal to 9, or alternatively greater than or equal to 5 and less than or equal to 6.

In one embodiment of the first aspect of the invention, adjusting the pH of the first evaporation concentrate comprises adding a pH adjuster selected from the group consisting of potassium hydroxide, sodium hydroxide, dipotassium phosphate, phosphoric acid, hydrochloric acid, lactic acid, and combinations thereof to the first evaporation concentrate.

In one embodiment of the first aspect of the invention, wherein the at least one enzyme added to the pH-adjusted first evaporation concentrate comprises one or more beta-lactase.

In one embodiment of the first aspect of the invention, the method further comprises deactivating the at least one enzyme by heating the first reaction product to a first threshold temperature, wherein the at least one enzyme is inactive in the first reaction product. In one such embodiment, the first threshold temperature is greater than or equal to 80 degrees Celsius.

In one embodiment of the first aspect of the invention, the second ultrafiltration step comprises filtering the deactivated evaporation concentrate through an ultrafilter having an average pore size of between 5,000 Daltons and 10,000 Daltons.

In one embodiment of the first aspect of the invention, the method does not comprise deactivating the at least one enzyme by heating the first reaction product to a first threshold temperature, and wherein the enzymes collected in the second ultrafiltration retentate are recycled for use in the first reaction step.

In one embodiment of the first aspect of the invention, the dairy-derived galacto-oligosaccharide product comprises a proximate composition comprising between 60% DMB and 75% DMB the galacto-oligosaccharide, less than or equal to 20% DMB lactose, less than or equal to 10% DMB glucose, and less than or equal to 10% DMB galactose.

In one embodiment of the first aspect of the invention, the method further includes subjecting the dairy-derived galacto-oligosaccharide product to a drying step.

In a second aspect of the invention, the method includes: subjecting a pasteurized whey mixture having an original concentration of lactose to a first ultrafiltration step to form a first ultrafiltration retentate and a first ultrafiltration permeate; subjecting the first ultrafiltration permeate to a separation step to form a first separation permeate and a first separation retentate, wherein the first separation step consists of either a nanofiltration separation or a reverse osmosis separation; subjecting the first separation retentate to a first evaporation step to form a first evaporation concentrate and a first evaporation condensate; adjusting a pH of the first evaporation concentrate to a value greater than or equal to 3 and less than or equal to 9 to form a pH-adjusted first evaporation concentrate; reacting, to form a galacto-oligosaccharide, at least one enzyme with the first evaporation concentrate by adding the at least one enzyme to the first evaporation concentrate to form a first reaction product comprising the galacto-oligosaccharide; subjecting the first reaction product to a second ultrafiltration step to form a second ultrafiltration retentate and a second ultrafiltration permeate, the second ultrafiltration permeate being poorer in a protein concentration than the second ultrafiltration retentate; subjecting the second ultrafiltration permeate to a second ion exchange chromatography step comprising reducing one or more mineral concentrations in the second ultrafiltration permeate to form a second mineral-reduced mixture; subjecting the second mineral-reduced mixture to a second evaporation step to form a second evaporation concentrate and a second evaporation condensate; separating a portion of the second evaporation concentrate to form a second evaporation concentrate bypass mixture and a second evaporation concentrate line mixture; subjecting the second evaporation concentrate line mixture to a fractionation chromatography step to form a first fractionation portion and a second fractionation portion, wherein the first fractionation portion comprises the galacto-oligosaccharide; subjecting the first fractionation portion to a third evaporation step to form a third evaporation concentrate and a third evaporation condensate; mixing the third evaporation concentrate and the second evaporation concentrate bypass mixture to form a premium mixture; subjecting the premium mixture to a polishing ion exchange chromatography step to form a polished premium mixture; subjecting the polished premium mixture to a fourth evaporation step to form a fourth evaporation condensate and a fourth evaporation concentrate, the fourth evaporation concentrate comprising the dairy-derived galacto-oligosaccharide product, the dairy-derived galacto-oligosaccharide product comprising lactose in a concentration less than the original concentration of lactose.

In one embodiment of the second aspect of the invention, the method further comprises, prior to subjecting the pasteurized whey mixture to the first ultrafiltration step, pasteurizing a clarified whey mixture comprising a dairy-derived acid whey and the original concentration of lactose, to form the pasteurized whey mixture. In a further embodiment thereof, pasteurizing the clarified whey mixture comprises subjecting the clarified whey mixture to a high-temperature, short-time pasteurization process, wherein the high-temperature, short-time pasteurization process is conducted at a temperature greater than or equal to 161° F. and for a duration greater than or equal to 15 seconds.

In one embodiment of the second aspect of the invention, the first ultrafiltration step comprises filtering the pasteurized whey mixture through an ultrafilter having an average pore size of between 5,000 Daltons and 10,000 Daltons.

In one embodiment of the second aspect of the invention, the first separation step consists of a nanofiltration separation, wherein the nanofiltration separation comprises filtering the ultrafiltration permeate through a nanofilter having an average pore size of between 150 Daltons and 300 Daltons.

In one embodiment of the second aspect of the invention, the method further comprises, prior to the first evaporation step, subjecting the first separation retentate to a first electrodialysis step comprising reducing one or more mineral concentrations in the first separation retentate. In one such embodiment, the one or more mineral concentrations reduced in the first electrodialysis step comprise a mineral selected from a group consisting of calcium, magnesium, phosphorus, potassium, sodium, and combinations thereof.

In one embodiment of the second aspect of the invention, the method further comprises, prior to the first evaporation step, subjecting the first separation retentate to a first ion exchange chromatography step comprising reducing one or more mineral concentrations in the first separation retentate. In one such embodiment, the one or more mineral concentrations reduced in the first ion exchange chromatography step comprise a mineral selected from a group consisting of calcium, magnesium, phosphorus, potassium, sodium, and combinations thereof.

In one embodiment of the first aspect of the invention, adjusting the pH of the first evaporation concentrate comprises adjusting the pH to a value in a pH range selected from the group consisting of greater than or equal to 3 and less than or equal to 7, alternatively greater than or equal to 5 and less than or equal to 9, or alternatively greater than or equal to 5 and less than or equal to 6.

In one embodiment of the second aspect of the invention, adjusting the pH of the first evaporation concentrate comprises adding a pH adjuster selected from the group consisting of potassium hydroxide, sodium hydroxide, dipotassium phosphate, phosphoric acid, hydrochloric acid, lactic acid, and combinations thereof to the first evaporation concentrate.

In one embodiment of the second aspect of the invention, wherein the at least one enzyme added to the pH-adjusted first evaporation concentrate comprises one or more beta-lactase.

In one embodiment of the second aspect of the invention, the method further comprises deactivating the at least one enzyme by heating the first reaction product to a first threshold temperature, wherein the at least one enzyme is inactive in the first reaction product. In one such embodiment, the first threshold temperature is greater than or equal to 80 degrees Celsius.

In one embodiment of the second aspect of the invention, the second ultrafiltration step comprises filtering the deactivated evaporation concentrate through an ultrafilter having an average pore size of between 5,000 Daltons and 10,000 Daltons.

In one embodiment of the second aspect of the invention, the method does not comprise deactivating the at least one enzyme by heating the first reaction product to a first threshold temperature, and wherein the enzymes collected in the second ultrafiltration retentate are recycled for use in the first reaction step.

In one embodiment of the second aspect of the invention, the dairy-derived galacto-oligosaccharide product comprises a proximate composition comprising less than or equal to 85% DMB galacto-oligosaccharide, less than or equal to 5% DMB lactose, less than or equal to 2% DMB glucose, and less than or equal to 2% DMB galactose.

In one embodiment of the second aspect of the invention, the method further includes subjecting the dairy-derived galacto-oligosaccharide product to a drying step.

In a third aspect of the invention, the method includes: subjecting a pasteurized whey mixture having an initial concentration of lactose to a first ultrafiltration step to form a first ultrafiltration retentate and a first ultrafiltration permeate; subjecting the first ultrafiltration permeate to a first separation step to form a first separation permeate and a first separation retentate, wherein the first separation step consists of either a nanofiltration separation or a reverse osmosis separation; subjecting the first separation retentate to a first evaporation step to form a first evaporation concentrate and a first evaporation condensate; adjusting a pH of the first evaporation concentrate to a value greater than or equal to 3 and less than or equal to 9 to form a pH-adjusted first evaporation concentrate; reacting, to form a galacto-oligosaccharide, a first at least one enzyme with the first evaporation concentrate by adding the at least one enzyme to the first evaporation concentrate to form a first reaction product comprising the galacto-oligosaccharide; subjecting the first reaction product to a second ultrafiltration step to form a second ultrafiltration retentate and a second ultrafiltration permeate, the second ultrafiltration permeate being poorer in a protein concentration than the second ultrafiltration retentate; subjecting the second ultrafiltration permeate to a second ion exchange chromatography step comprising reducing one or more mineral concentrations in the second ultrafiltration permeate to form a second mineral-reduced mixture; subjecting the second mineral-reduced mixture to a second evaporation step to form a second evaporation concentrate and a second evaporation condensate; separating a portion of the second evaporation concentrate to form a second evaporation concentrate bypass mixture and a second evaporation concentrate line mixture; subjecting the second evaporation concentrate line mixture to a fractionation chromatography step to form a first fractionation portion and a second fractionation portion, wherein the first fractionation portion comprises the galacto-oligosaccharide; subjecting the first fractionation portion to a third evaporation step to form a third evaporation concentrate and a third evaporation condensate; mixing the third evaporation concentrate and the second evaporation concentrate bypass mixture to form a premium mixture; cooling the premium mixture to form a cooled premium mixture; reacting, to form additional galacto-oligosaccharide, a second at least one enzyme with the cooled premium mixture to form an enzyme premium mixture; subjecting the enzyme premium mixture to a third ultrafiltration step to form a third ultrafiltration permeate and a third ultrafiltration retentate; subjecting the third ultrafiltration permeate to a third ion exchange chromatography step to form a super-premium mixture; subjecting the super-premium mixture to a polishing ion exchange chromatography step to form a polished super-premium mixture; subjecting the polished super-premium mixture to a fourth evaporation step to form a fourth evaporation condensate and a fourth evaporation concentrate, the fourth evaporation concentrate comprising the dairy-derived galacto-oligosaccharide product, the dairy-derived galacto-oligosaccharide product comprising lactose in a concentration less than the original concentration of lactose.

In one embodiment of the third aspect of the invention, the method further comprises, prior to subjecting the pasteurized whey mixture to the first ultrafiltration step, pasteurizing a clarified whey mixture comprising a dairy-derived acid whey and the original concentration of lactose, to form the pasteurized whey mixture. In a further embodiment thereof, pasteurizing the clarified whey mixture comprises subjecting the clarified whey mixture to a high-temperature, short-time pasteurization process, wherein the high-temperature, short-time pasteurization process is conducted at a temperature greater than or equal to 161° F. and for a duration greater than or equal to 15 seconds.

In one embodiment of the third aspect of the invention, the first ultrafiltration step comprises filtering the pasteurized whey mixture through an ultrafilter having an average pore size of between 5,000 Daltons and 10,000 Daltons.

In one embodiment of the third aspect of the invention, the first separation step consists of a nanofiltration separation, wherein the nanofiltration separation comprises filtering the ultrafiltration permeate through a nanofilter having an average pore size of between 150 Daltons and 300 Daltons.

In one embodiment of the third aspect of the invention, the method further comprises, prior to the first evaporation step, subjecting the first separation retentate to a first electrodialysis step comprising reducing one or more mineral concentrations in the first separation retentate. In one such embodiment, the one or more mineral concentrations reduced in the first electrodialysis step comprise a mineral selected from a group consisting of calcium, magnesium, phosphorus, potassium, sodium, and combinations thereof.

In one embodiment of the third aspect of the invention, the method further comprises, prior to the first evaporation step, subjecting the first separation retentate to a first ion exchange chromatography step comprising reducing one or more mineral concentrations in the first separation retentate. In one such embodiment, the one or more mineral concentrations reduced in the first ion exchange chromatography step comprise a mineral selected from a group consisting of calcium, magnesium, phosphorus, potassium, sodium, and combinations thereof.

In one embodiment of the third aspect of the invention, adjusting the pH of the first evaporation concentrate comprises adjusting the pH to a value in a pH range selected from the group consisting of greater than or equal to 3 and less than or equal to 7, alternatively greater than or equal to 5 and less than or equal to 9, or alternatively greater than or equal to 5 and less than or equal to 6.

In one embodiment of the third aspect of the invention, adjusting the pH of the first evaporation concentrate comprises adding a pH adjuster selected from the group consisting of potassium hydroxide, sodium hydroxide, dipotassium phosphate, phosphoric acid, hydrochloric acid, lactic acid, and combinations thereof to the first evaporation concentrate.

In one embodiment of the third aspect of the invention, wherein the at least one enzyme added to the pH-adjusted first evaporation concentrate comprises one or more beta-lactase.

In one embodiment of the third aspect of the invention, the method further comprises deactivating the first at least one enzyme by heating the first reaction product to a first threshold temperature, wherein the at least one enzyme is inactive in the first reaction product. In one such embodiment, the first threshold temperature is greater than or equal to 80 degrees Celsius.

In one embodiment of the third aspect of the invention, the method does not comprise deactivating the first at least one enzyme by heating the first reaction product to a first threshold temperature, and wherein the enzymes collected in the second ultrafiltration retentate are recycled for use in the first reaction step, the second reaction step, or both.

In one embodiment of the third aspect of the invention, the method further comprises deactivating the second at least one enzyme by heating the enzyme premium mixture to a first threshold temperature, wherein the second at least one enzyme is inactive in the enzyme premium mixture. In one such embodiment, the first threshold temperature is greater than or equal to 80 degrees Celsius.

In one embodiment of the third aspect of the invention, the method does not comprise deactivating the second at least one enzyme by heating the enzyme premium mixture to a first threshold temperature, and wherein the enzymes collected in the third ultrafiltration retentate are recycled for use in the first reaction step, the second reaction step, or both.

In one embodiment of the third aspect of the invention, the second ultrafiltration step comprises filtering the deactivated evaporation concentrate through an ultrafilter having an average pore size of between 5,000 Daltons and 10,000 Daltons.

In one embodiment of the third aspect of the invention, the dairy-derived galacto-oligosaccharide product comprises a proximate composition comprising less than or equal to 90% DMB galacto-oligosaccharide, less than or equal to 0.01% DMB lactose, less than or equal to 0.01% DMB glucose, and less than or equal to 2% DMB galactose.

In one embodiment of the third aspect of the invention, the method further includes subjecting the dairy-derived galacto-oligosaccharide product to a drying step.

### BRIEF DESCRIPTION OF THE DRAWINGS

Further features, advantages and possible applications of the present invention will be apparent from the following description in connection with the figures, in which the same reference signs are used throughout for the same or mutually corresponding elements of the invention. The accompanying drawings, which are incorporated in and constitute a part of this specification, illustrate one or more embodiments of the invention and, together with the general description given above and the detailed description given below, explain the one or more embodiments of the invention.

FIG. 1 shows an embodiment of a first portion of a process flow diagram for methods of manufacturing a dairy-derived galacto-oligosaccharide product.

FIG. 2A shows an embodiment of a second portion of a process flow diagram for a method of manufacturing a dairy-derived galacto-oligosaccharide product.

FIG. 2B shows another embodiment of a second portion of a process flow diagram for a method of manufacturing a dairy-derived galacto-oligosaccharide product.

FIG. 2C shows a further embodiment of a second portion of a process flow diagram for a method of manufacturing a dairy-derived galacto-oligosaccharide product.

### DETAILED DESCRIPTION

According to embodiments of the invention summarized herein, the methods form a dairy-derived galacto-oligosaccharide product. The methods described herein form a dairy-derived galacto-oligosaccharide product, which may be used in other products.

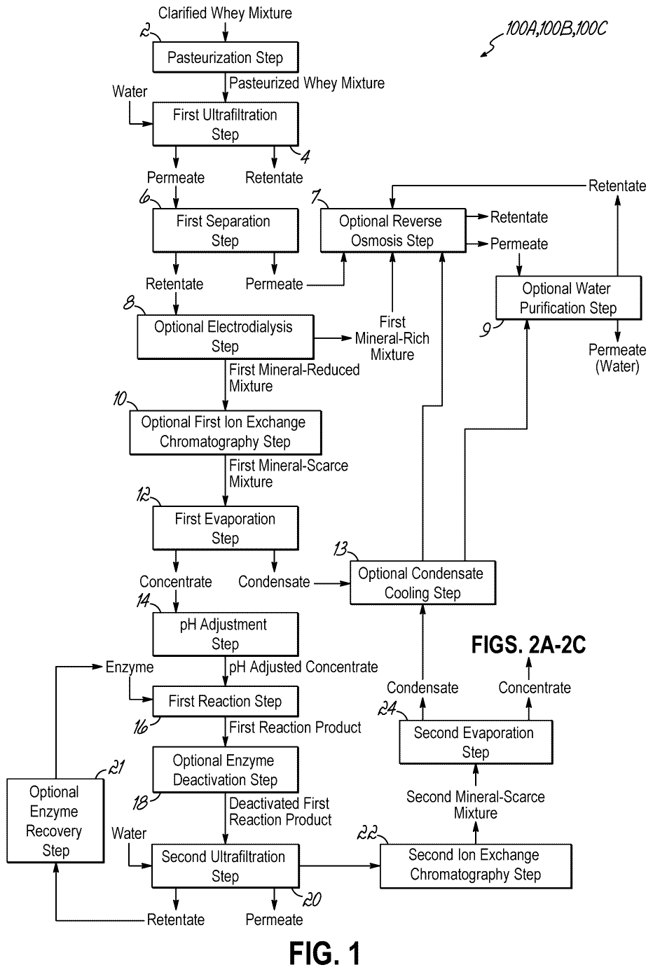

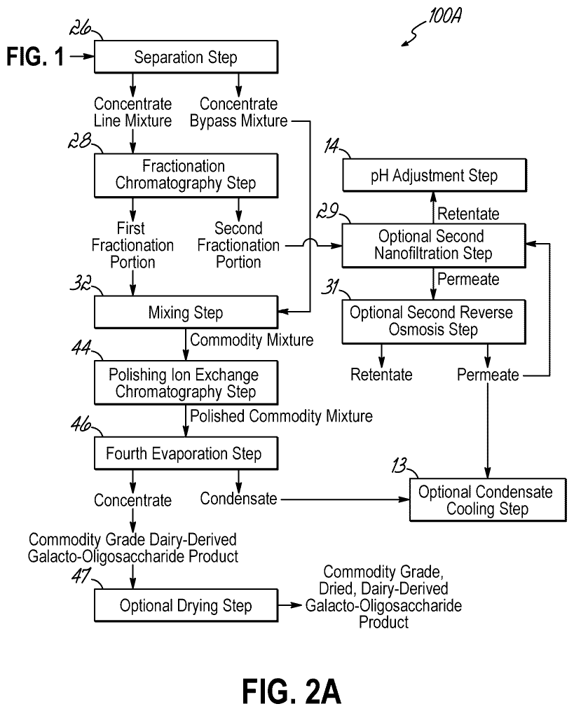

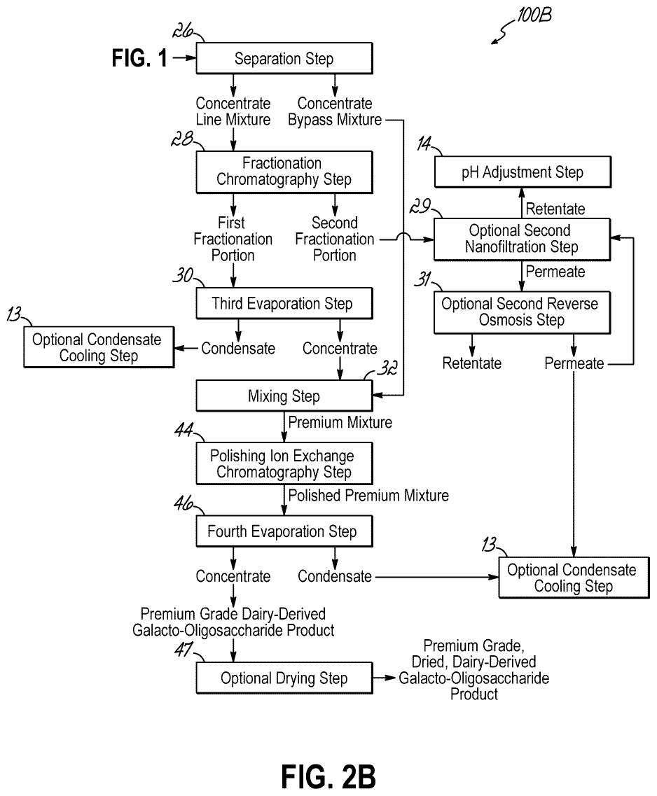

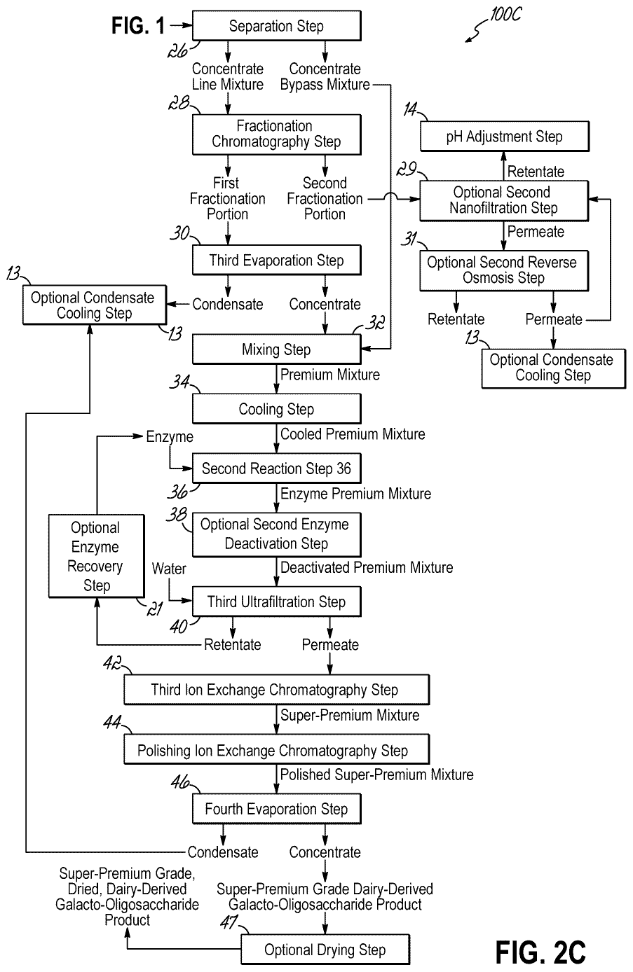

FIGS. 1 and 2A-2C, together, show three methods 100A, 100B, and 100C of making a dairy-derived galacto-oligosaccharide product. FIG. 1 shows a portion of methods 100A, 100B, and 100C that are the same between all methods while FIGS. 2A-2C show portions of the methods 100A, 100B, and 100C that are specific to their respective methods. Accordingly, steps having the same numbers in FIGS. 2A-2C should be understood to be implemented in the same manner unless otherwise indicated. Several steps and/or processes included in the method 100A, 100B, 100C are included to reduce/remove contaminants such as undesired fats, proteins, or minerals from the streams that will ultimately become the dairy-derived galacto-oligosaccharide product.

Referring now to FIG. 1, the first portion of methods 100A, 100B, and 100C for manufacturing a dairy-derived galacto-oligosaccharide product are shown. The method 100A, 100B, 100C includes subjecting a clarified whey mixture to a pasteurization step 2 to form a pasteurized whey mixture. The clarified whey mixture may be or comprise any whey product formed from a dairy-related manufacturing process, for example, but not limited to, acid whey which forms as a byproduct from dairy-derived yogurt manufacturing or Greek yogurt manufacturing. In one embodiment, the clarified whey mixture is a mixture of various proteins and carbohydrates, and includes at least an original concentration of lactose. Prior to introduction into, or during, the method 100A, 100B, 100C described herein, the acid whey may be clarified using conventional clarification processes to form the clarified whey mixture.

The pasteurization step 2 may comprise subjecting the clarified whey mixture to a high-temperature short-time (HTST) process to form the pasteurized whey mixture. In another embodiment, the pasteurization step 2 includes subjecting the clarified whey mixture to a plurality of HTST processes in parallel. In a preferred embodiment thereof, the pasteurization step 2 of method 100A, 100B, 100C comprises two high-temperature short-time processes ran in parallel into which the clarified whey is fed. In yet another embodiment, the pasteurization step 2 includes subjecting the clarified whey mixture to a plurality of HTST processes in sequence. In embodiments involving either parallel or sequential HTST processes, a greater plurality than 2 may be used. In embodiments of the pasteurization step 2 comprising an HTST process, the clarified whey is heated to reduce and/or destroy pathogens that may have been present in the clarified whey. In one embodiment, the clarified whey is heated to a temperature and duration sufficient for pasteurization. In a preferred embodiment, the clarified whey is heated to a temperature greater than or equal to 161° F. for a duration greater than or equal to 15 seconds to pasteurize the clarified whey and form the pasteurized whey mixture.

Following the pasteurization step 2, at least a portion of the pasteurized whey mixture is subjected to a first ultrafiltration step 4 to form a first ultrafiltration permeate and a first ultrafiltration retentate. In one embodiment, the entirety of the pasteurized whey mixture is subjected to the first ultrafiltration step. Water may optionally be added to the pasteurized whey mixture prior to the first ultrafiltration step 4, during the first ultrafiltration step 4, or both to enable diafiltration. The first ultrafiltration step 4 includes subjecting the pasteurized whey mixture through one or more filtration sieve(s) to separate large molecules, such as fats and/or proteins, initially present in the pasteurized whey mixture into the first ultrafiltration retentate from smaller molecules, such as carbohydrates, organic acids, minerals, water, etc., initially included in the pasteurized whey mixture into the first ultrafiltration permeate. In one embodiment, the one or more filtration sieve(s) used in the ultrafiltration step 4 have an average pore size greater than or equal to about 5,000 Daltons, alternatively less than or equal to 10,000 Daltons, or alternatively between about 5,000 Daltons and about 10,000 Daltons (i.e., greater than or equal to about 5,000 Daltons and less than or equal to about 10,000 Daltons). The larger molecules separated in this way are included in the first ultrafiltration retentate, which may be drained from the method 100A, 100B, 100C and may be stored or further processed into other dairy products.

Following the first ultrafiltration step 4, at least a portion of the first ultrafiltration permeate is subjected to a first separation step 6 to form a first separation retentate and a first separation permeate. In one embodiment, the entirety of the first ultrafiltration permeate is subjected to the first separation step 6. The first separation step 6 may include a reverse osmosis (RO) process, a nanofiltration process, or both. In embodiments including an RO process, the water is removed as the first separation permeate and the remaining solids comprise the first separation retentate. In one such embodiment, the first separation step 6 includes subjecting the entirety of the first ultrafiltration retentate to the reverse osmosis process. In another embodiment, the first separation step 6 includes subjecting the first ultrafiltration retentate through one or more filtration sieve(s) to separate large solids initially included in the first ultrafiltration retentate into the first separation retentate from small minerals originally included in the first ultrafiltration retentate into the first separation permeate. In one embodiment, the one or more filtration sieve(s) used in the first separation step 6 have an average pore size greater than or equal to about 150 Daltons, alternatively less than or equal to about 300 Daltons, or alternatively between about 150 Daltons and about 300 Daltons. In some embodiments, at least a portion of the first separation permeate may be subjected to an optional reverse osmosis step 7 (discussed in further detail below).

Following the first separation step 6, at least a portion of the first separation retentate may be subjected to an optional electrodialysis step 8 for further separation of minerals from the first separation retentate to form a first mineral-reduced mixture (which may be referred to in the claims as the first separation retentate in embodiments including this optional step) and a first mineral-rich mixture. In one embodiment, the entirety of the first separation retentate is subjected to the electrodialysis step 8. In one embodiment, the electrodialysis step 8 involves subjecting the first separation retentate to a plurality electrodialysis processes ran in parallel, in sequence, or both. In a preferred embodiment of the electrodialysis step 8, two electrodialysis processes are ran in parallel. In another embodiment, only one electrodialysis process is used in the electrodialysis step 8. The electrodialysis step 8 is configured to separate the first separation retentate, by electrodialysis, into a mineral-rich mixture, and a mineral-reduced mixture. The mineral-rich mixture includes a greater percentage of minerals included in the first separation retentate that is subjected to the electrodialysis step 8 compared to the mineral-reduced mixture. In some embodiments, at least a portion of the mineral-rich mixture may be further processed in an optional reverse osmosis step 7 (discussed further below).

In some embodiments, at least a portion of the mineral-reduced mixture, or at least a portion of the first separation retentate, may be stored in an optional buffer tank (not shown) prior to optionally subjecting the mineral reduced mixture or the first separation retentate to an optional first ion exchange chromatography step 10. The optional buffer tank may not be used or needed in all embodiments of the invention, especially in a steady-state manufacturing process in which storage of intermediate compositions is not needed. However, in embodiments where the optional buffer tank is used, it may be a tank specifically designed to isolate the mineral-reduced mixture from outside influence, and thus allow for the isolated storage of the mineral-reduced mixture prior to being subjected to the first ion exchange chromatography step 10.

Generally speaking, in embodiments implementing the optional electrodialysis step 8, some portion of the undesired components present in the first separation retentate, such as one or more ions and/or minerals included in the first separation retentate, are removed from the mineral-reduced mixture into the mineral-rich mixture while leaving lactose, other small carbohydrates, and/or water in the mineral-reduced mixture. However, it is possible that some amount of the undesirable components present in the first separation retentate remain in the mineral-reduced mixture. In some embodiments, the concentration(s) of one or more ions and/or minerals present in the first separation retentate is reduced during the first electrodialysis step 8. For example, the concentration(s) of the one or more ions and/or minerals reduced in this way may be an ion or mineral selected from the group consisting of calcium, magnesium, phosphorus, potassium, sodium, and combinations thereof. In one such embodiment, the total concentration of ions and/or minerals in the mineral-reduced mixture is less than or equal to 50% of the initial concentration of the ions and/or minerals in the first separation retentate, alternatively less than or equal to 25% of the initial concentration of the first separation retentate, alternatively less than or equal to 20% of the initial concentration of the first separation retentate, alternatively less than or equal to 15% of the initial concentration of the first separation retentate, alternatively less than or equal to 10% of the initial concentration of the first separation retentate, alternatively less than or equal to 5% of the initial concentration of the first separation retentate, or alternatively less than or equal to 1% of the initial concentration of the first separation retentate.

Following the optional electrodialysis step 8, or following the first separation step 6 in embodiments not including the optional electrodialysis step 8, at least a portion of the mineral-reduced mixture may be further subjected to an optional first ion exchange chromatography step 10 to produce a mineral-scarce mixture. In one such embodiment, the entirety of the mineral-reduced mixture is subjected to the first ion-exchange chromatography step 10. In another such embodiment, the optional electrodialysis step 8 is not included and the entirety of the first separation retentate is subjected to the first ion-exchange chromatography step 10. in embodiments when it is included, the first ion exchange chromatography step 10 is configured to reduce or further reduce the concentration of the one or more ions and/or minerals in the first separation retentate and/or mineral-reduced mixture (i.e., demineralize the first separation retentate and/or mineral-reduced mixture) to form a first mineral-scarce mixture. In one embodiment, the first ion exchange chromatography step 10 separates ions of different charges included in the mineral-reduced mixture to form the first mineral-scarce mixture. In embodiments including both step 8 and step 10, the first mineral-scarce mixture includes even fewer minerals than the mineral-reduced mixture, thus the mineral-reduced mixture is further purified to form the first mineral-scarce mixture. In one embodiment, the first ion exchange chromatography step 10 is configured to remove components that contribute to undesirable colors. In some embodiments, the concentrations of the one or more ions and/or minerals in the mineral-reduced mixture is reduced during the first ion exchange chromatography step 10. For example, the one or more ion concentrations or mineral concentrations reduced in this way may be an ion or mineral selected from a group consisting of calcium, magnesium, phosphorus, potassium, sodium, and combinations thereof.

In some embodiments, at least a portion of the first separation retentate, the first mineral-reduced mixture, and/or the first mineral-scarce mixture may be stored in an optional ion exchange outlet tank (not shown) prior to a first evaporation step 12. In one embodiment, the entirety of the first separation retentate, the first mineral-reduced mixture, and/or the first mineral-scarce mixture may be stored in an optional ion exchange outlet tank prior to the first evaporation step 12. The optional ion exchange outlet tank may be, by way of example and not limitation, a buffer tank or a storage tank. In one embodiment, the optional ion exchange outlet tank is a storage tank. In one embodiment, the optional ion exchange outlet tank is a buffer tank. In one embodiment, the temperature of the first separation retentate, the first mineral-reduced mixture, and/or the first mineral-scarce mixture is adjusted in the optional ion exchange outlet tank prior to the first evaporation step 12. In another embodiment, a pH adjuster is added to the first separation retentate, the first mineral-reduced mixture, and/or the first mineral-scarce mixture in the optional ion exchange outlet tank. In some embodiments, the optional ion exchange outlet tank may not be used or needed, such as in embodiments of the invention involving a steady-state manufacturing process in which storage of intermediate compositions is not needed.

After the first ion exchange chromatography step 10 in embodiments of the invention including this optional step, at least a portion of the first mineral-scarce mixture is subjected to a first evaporation step 12 to form a first evaporation condensate and a first evaporation concentrate. Alternatively, in embodiments not including optional step 10 but including step 8, at least a portion of the first mineral-reduced mixture is subjected to the first evaporation step 12. Alternatively, in embodiments not including either optional steps 8 or 10, at least a portion of the first separation retentate is subjected to the first evaporation step 12. In one embodiment, the entirety of the first mineral-scarce mixture, first mineral-reduced mixture, or first separation retentate is subjected to the first evaporation step 12. In some embodiments, the first evaporation condensate may be subjected to an optional condensate cooling step 13 to form a cooled condensate (discussed further below) configured to recycle water for use in one or more steps in the system. In other embodiments, an alternative means of removing water from the system may be used instead of evaporation in the first evaporation step 12 such as, by way of example and not limitation, an RO process.

With further reference to optional steps including the optional reverse osmosis step 7, an optional water purification step 9 (discussed further below), and the optional condensate cooling step 13, one or more of these steps may be configured to recycle water and/or remove undesirable components from the system for collection or further processing into other products not described herein. Looking first to the optional reverse osmosis step 7, the first separation permeate, the first mineral rich mixture, the cooled condensate, or a combination thereof may be subjected to a reverse osmosis process to form a reverse osmosis permeate and a reverse osmosis retentate. In one embodiment, at least a portion of the reverse osmosis retentate is removed from further processing in the methods 100A, 100B, and 100C. In one embodiment, the entirety of the reverse osmosis retentate is removed from further processing in the methods 100A, 100B, and 100C. In one embodiment, at least a portion of the reverse osmosis retentate is collected for further processing and/or to produce another product not described herein. In one embodiment, the entirety of the reverse osmosis retentate is collected for further processing and/or to produce another product not described herein.

With respect to the optional water purification step 9, in one embodiment, at least a portion of the reverse osmosis permeate, the entirety of the reverse osmosis permeate, at least a portion of the cooled condensate, the entirety of the cooled condensate, or a combination thereof is subjected to the optional water purification step 9 to produce a purified water permeate and a purified water retentate. In one embodiment, the purified water retentate is subjected to the optional reverse osmosis step 7 for further processing. In another embodiment, the purified water retentate is removed from the system and/or collected for further processing into another product not described herein. In one embodiment, the purified water permeate formed in the optional water purification step 9 may be used as water added into any step of methods 100A, 100B, or 100C involving the addition of water, such as the first ultra filtration step 4, the second ultrafiltration step 20 (discussed further below), the third ultrafiltration step (discussed further below), or a combination thereof.

With respect to the optional condensate cooling step 13, in one embodiment, one or more permeate or condensate stream is cooled to a temperature greater than or equal to 40° C., alternatively less than or equal to 60° C., or still alternatively between 40° C. and 60° C. to form the cooled condensate. As described above, at least a portion of the first evaporation condensate formed from the first evaporation step 12, or alternatively the entirety of the first evaporation condensate, may be subjected to the optional condensate cooling step 13. However, additionally or alternatively, one or more other condensate and/or permeate stream defined in later steps of the methods 100A, 100B, or 100C may be subjected to the optional condensate cooling step 13. In one such embodiment, at least a portion of one or more condensate and/or permeate streams selected from the list consisting of the second evaporation step condensate defined in the second evaporation step 24 (discussed further below), the third evaporation condensate stream defined in the third evaporation step 30 (described further below), the reverse osmosis permeate stream defined in the optional reverse osmosis step 31 (described further below), the fourth evaporation condensate stream defined in the fourth evaporation step 46 (described further below, or a combination thereof is subjected to the optional condensate cooling step 13. In another embodiment, the entirety of one or more condensate and/or permeate streams selected from the list consisting of the second evaporation step condensate defined in the second evaporation step 24 (discussed further below), the third evaporation condensate stream defined in the third evaporation step 30 (described further below), the reverse osmosis permeate stream defined in the optional reverse osmosis step 31 (described further below), the fourth evaporation condensate stream defined in the fourth evaporation step 46 (described further below, or a combination thereof is subjected to the optional condensate cooling step 13.

After the first evaporation step 12, at least a portion of the first evaporation concentrate is subjected to the pH adjustment step 14 to form the pH adjusted concentrate. In one embodiment, the entirety of the first evaporation concentrate is subjected to the pH adjustment step. In one embodiment, the pH adjustment step 14 involves adding a pH adjuster to the first evaporation concentrate. The pH adjuster can either increase or decrease the pH of the first evaporation concentrate as desired by adding either an acid or a base to the first evaporation concentrate and forming a pH-adjusted first evaporation concentrate. In one embodiment, the pH adjustment step is configured to add the pH adjuster in an amount sufficient to adjust the pH of the first evaporation concentrate to a target pH. In one such embodiment, the target pH for the pH-adjusted first evaporation concentrate is greater than or equal to 3 and less than or equal to 9, alternatively greater than or equal to 3 and less than or equal to 7, alternatively greater than or equal to 5 and less than or equal to 9, or alternatively greater than or equal to 5 and less than or equal to 6. In further embodiments thereof, the pH range selected may depend on the enzyme used in the later first reaction step 16 (discussed further below). For example, if the first reaction step 16 uses an enzyme selected from the list consisting of Nola Fiber, Nola GOS, Maxilact A4, and a combination thereof, it is preferable to maintain the pH in a range between 3 and 7. In another example, if the first reaction step 16 (discussed further below) uses the Maxilact Lgi 5000 enzyme, it is preferable to maintain the pH in a range between 5 and 9. Other enzymes that can be used in this process may have different preferred pH ranges, so these disclosures are not intended to be limiting. Non-limiting examples of pH adjusters that may be used in the pH adjusting step 14 include those selected from the group consisting of potassium hydroxide, sodium hydroxide, dipotassium phosphate, phosphoric acid, hydrochloric acid, lactic acid, and combinations thereof.

Following the pH adjustment step 14, at least a portion of the pH adjusted concentrate is subjected to a reaction step 16 to form a first reaction product. In one embodiment, the reaction step 16 comprises an enzyme reaction, whereby the enzyme reaction occurs by adding at least one enzyme to the pH adjusted concentrate to form a first reaction product, which includes the galacto-oligosaccharide. As discussed above, the enzyme used in an enzyme reaction may be selected from the list of enzyme products consisting of Nola Fiber (supplied by CHR Hansen Inc., version 1, including 5-10% beta-galactosidase), Nola GOS (supplied by Kerry Ingredients & Flavours Ltd.), Maxilact A4 (supplied by DSM Food Specialties B.V., including 1-10% beta-galactosidase), Maxilact Lgi 5000 (supplied by DSM Food Specialties B.V., including 1-10% beta-galactosidase), other suitable enzyme products including a beta-lactase, or a combination thereof. The at least one enzyme added during an enzyme reaction step 16 may include, but is not limited to, one or more beta-lactase (also known as beta-galactosidase), which may come from a variety of sources. For example, the at least one enzyme added during an enzyme reaction step 16 may come from Bifidobacterium bifidum, Aspergillus niger, Kluyveromyces lactis, or combinations thereof. In one embodiment, the at least one enzyme added during the enzyme reaction includes beta-galactosidase (lactase) from Bifidobacterium bifidum at 1.5% of lactose content by mass. Alternatively, or in addition, the at least one enzymes added during the enzyme reaction step 16 may include beta-galactosidase from Aspergillus niger at 1.5% of lactose content by mass. Yet further alternatively, or yet further in addition to the above, the at least one enzyme added during the enzyme reaction step 16 may include Aspergillus niger at 0.75% lactose content by mass and Kluyveromyces lactis at 0.75% lactose content by mass. In one embodiment, the amount of the at least one enzyme may be greater than or equal to 0.25% lactose content by mass and less than or equal to 3% lactose content by mass, or alternatively greater than or equal to 0.25% lactose content by mass and less than or equal to 2% lactose content by mass, alternatively greater than or equal to 0.25% lactose content by mass and less than or equal to 1% lactose content by mass, alternatively greater than or equal to 1% lactose content by mass and less than or equal to 3% lactose content by mass, or alternatively greater than or equal to 1% lactose content by mass and less than or equal to 2% lactose content by mass.

In one embodiment of the reaction step 16, a specific combination of enzymes is used including a lactose oxidase and/or a catalase in addition to the beta-galactosidase. In one such embodiment, the lactose oxidase is included in an amount greater than or equal to about 1% by mass and less than or equal to about 3% by mass. In one such embodiment, the catalase is included in an amount greater than or equal to about 0.75% by mass and less than or equal to 1% by mass. The catalase, when used, may come from the enzyme product Catazyme (produced by CHR Hansen Inc., version 6, including 1-5% catalase). The lactose oxidase, when used, may come from the enzyme product LactoYIELD (produced by CHR Hansen Inc., version 8). In one embodiment, both Catazyme and LactoYIELD are used together.

Additionally or alternatively, glucose oxidase may be included to reduce the amount of glucose in the final product, which may increase the purity thereof. In one such embodiment, the amount of glucose oxidase is greater than or equal to about 1% by mass and less than or equal to about 3% by mass. The glucose oxidase, when used, may come from the enzyme product (supplied by DSM Food Specialties B.V., including 1-10% glucose oxidase).

In some embodiments of enzyme reaction step 16, one or more buffering agent is included, wherein the one or more buffering agent may be selected from the list consisting of sodium hydroxide, potassium hydroxide, dipotassium phosphate, or a combination thereof.

When the enzyme reaction step 16 is completed, at least a portion of the first reaction product is subjected to an optional enzyme deactivation step 18 to form the deactivated first reaction product. In one embodiment, the reaction step 16 is completed when the initial lactose content is reduced by 50% concentration. In another embodiment, the reaction step 16 is complete when the concentration of free glucose is increased to the point of greater than or equal to 20% by weight, or alternatively to the point of greater than or equal to 25% by weight. In one embodiment, the entirety of the first reaction product is subjected to the optional enzyme deactivation step 18. In one embodiment, the enzyme deactivation step 18 comprises increasing the temperature of the first reaction product to a temperature greater than or equal to a first threshold temperature to form the deactivated first reaction product. Without being bound by theory, such embodiments of this enzyme deactivation step 18 work because when the at least one enzyme is subjected to a temperature above the first threshold temperature, the at least one enzyme is rendered inactive. Accordingly, the first threshold temperature may be any temperature, above which, the at least one enzyme used in the reaction step 16 is rendered inactive. In one embodiment, the first threshold temperature is 80° C. In embodiments not including the optional first enzyme deactivation step 18, the enzymes may be recycled in an optional enzyme recovery step 21 (discussed further below).

Following the optional enzyme deactivation step 18, at least a portion of the deactivated first reaction product is subjected to a second ultrafiltration step 20 to form a second ultrafiltration permeate and a second ultrafiltration retentate. Alternatively, following the first reaction step 16 in embodiments not including the optional enzyme deactivation step 18, the first reaction product is subjected to the second ultrafiltration step 20 to form a second ultrafiltration permeate and a second ultrafiltration retentate. In one embodiment, the entirety of the deactivated first reaction product or the first reaction product is subjected to the second ultrafiltration step 20. The second ultrafiltration step 20 includes subjecting the deactivated first reaction product or the first reaction product through one or more filtration sieves. In one embodiment, the one or more filtration sieves have an average pore size greater than or equal to 5,000 Daltons, alternatively less than or equal to 10,000 Daltons, or alternatively between 5,000 Daltons and 10,000 Daltons. Excess water may optionally be used to increase the flow of the deactivated first reaction product or the first reaction product through the one or more filtration sieves (e.g., to enable diafiltration). In one embodiment, the second ultrafiltration step 20 is configured to separate the one or more enzymes, which are relatively large compared to other components included in the first reaction product or the deactivated first reaction product, from the other components included in the first reaction product or the deactivated first reaction product. Optionally, in embodiments not including the optional enzyme deactivation step 18, at least a portion of the second ultrafiltration retentate, which contains the still active enzymes separated in this way, may be subjected to an optional enzyme recovery step 21 configured to recover the one or more enzymes for reuse in steps of the method 100A, 100B, or 100C wherein enzymes are added such as, for example, the first reaction step 16, the second reaction step 36, or a combination thereof. In one such embodiment, the entirety of the second ultrafiltration retentate is subjected to the optional enzyme recovery step 21. Alternatively, a portion of or the entirety of the second ultrafiltration retentate may be stored or collected for other uses not described herein.

Following the second ultrafiltration step 20, at least a portion of the second ultrafiltration permeate may be stored in an optional tank (not shown) before the second ultrafiltration permeate is eventually subjected to a second ion exchange chromatography step 22. In one such embodiment, the entirety of the second ultrafiltration permeate is stored in an optional tank for storage. In another embodiment, the optional tank is used as a buffer tank following the second ultrafiltration step 20. The optional tank may not be used or needed in all embodiments of the invention, especially in a steady-state manufacturing process in which storage of intermediate compositions is not needed.

Following the second ultrafiltration step 20, at least a portion of the second ultrafiltration permeate is subjected to a second ion exchange chromatography step 22 which further demineralizes and/or deionizes the second ultrafiltration permeate to form a second mineral-scarce mixture by separating out ions of different charges included in the second ultrafiltration permeate. In one embodiment, the entirety of the second ultrafiltration permeate is subjected to the second ion exchange chromatography step 22. In one embodiment, the second ion exchange chromatography step 22 is configured to remove mineral concentration remaining since the formation of the first mineral scarce mixture. In one embodiment, the second mineral-scarce mixture includes fewer minerals, ions, and/or impurities than the second ultrafiltration permeate. Without being bound by theory, intermediate steps between the first ion exchange chromatography step 10 and the second ion exchange chromatography step 22, such as the first reaction step 16, may increase the ion concentration and/or mineral concentration of the second ultrafiltration permeate relative to the first mineral scarce mixture. The second ion exchange chromatography step 22 also may remove components that contribute to undesirable colors. In some embodiments, one or more ion concentrations and/or mineral concentrations in the second ultrafiltration permeate is reduced during the second ion exchange chromatography step 22. For example, the one or more ion concentrations and/or mineral concentrations reduced in this way may be an ion or mineral selected from a group consisting of calcium, magnesium, phosphorus, potassium, sodium, and combinations thereof.

In some embodiments, at least a portion of the second mineral-scarce mixture may be stored in an optional ion exchange outlet tank (not shown) prior to subjecting the second mineral-scarce mixture to a second evaporation step 24. In one embodiment, the entirety of the second mineral-scarce mixture is stored in an optional ion exchange outlet tank prior to the second evaporation step. The optional ion exchange outlet tank may be, by way of example and not limitation, a buffer tank or a storage tank. In one embodiment, the optional ion exchange outlet tank is a storage tank. In one embodiment, the optional ion exchange outlet tank is a buffer tank. In one embodiment, the temperature of the second mineral-scarce mixture is adjusted in the optional ion exchange outlet tank prior to the first evaporation step 12. In another embodiment, a pH adjuster is added to the second mineral-scarce mixture in the optional ion exchange outlet tank. In some embodiments, the optional ion exchange outlet tank may not be needed or used, especially in a steady-state manufacturing process in which storage of intermediate compositions is not needed.

After the second ion exchange chromatography step 22, at least a portion of the second mineral-scarce mixture is subjected to a second evaporation step 24 to form a second evaporation condensate and a second evaporation concentrate. In one embodiment, the entirety of the second mineral-scarce mixture is subjected to the second evaporation step. In one embodiment, at least a portion of the second evaporation condensate may be cooled in optional cooling step 13 as described in more detail above. In another embodiment, the entirety of the second evaporation condensate is subjected to the optional condensate cooling step 13. The second evaporation concentrate proceeds on for further processing in methods 100A, 100B, and 100C as shown in FIGS. 100A, 100B, and 100C respectively. In other embodiments, an alternative means of removing water from the system may be used instead of evaporation in the second evaporation step 24 such as, by way of example and not limitation, an RO process.

There are varying grades of the dairy-derived galacto-oligosaccharide products that are able to be produced by the methods described herein. For example, a commodity grade product produced according to method 100A as shown in FIGS. 1 and 2A, a premium grade product produced according to method 100B as shown in FIGS. 1 and 2B, and a super premium grade product produced according to method 100C as shown in FIGS. 1 and 2C. The commodity grade dairy-derived galacto-oligosaccharide product includes a proximate composition comprising between 60% DMB and 75% DMB of the galacto-oligosaccharide, less than or equal to 20% DMB lactose, less than or equal to 10% DMB glucose, and less than or equal to 10% DMB galactose. The premium grade galacto-oligosaccharide product includes a proximate composition comprising less than or equal to 85% DMB galacto-oligosaccharide, less than or equal to 5% DMB lactose, less than or equal to 2% DMB glucose, and less than or equal to 2% DMB galactose. The super premium product includes a proximate composition comprising less than or equal to 90% DMB galacto-oligosaccharide, less than or equal to 0.01% DMB lactose, less than or equal to 0.01% DMB glucose, and less than or equal to 2% DMB galactose. Various combinations of the unit operations described herein are used to achieve the desired grade of galacto-oligosaccharide product.

With reference to FIG. 2A, which shows method 100A for producing the commodity grade product, after the process steps described above for FIG. 1 have been performed, at least a portion of the second evaporation concentrate is subjected to separation step 26 to form a second evaporation concentrate bypass mixture and a second evaporation concentrate line mixture. In one embodiment, the entirety of the second evaporation concentrate is subjected to the separation step 26. The separation step 26 may involve the use of one or more of the following pieces of equipment, including but not limited to valve clusters, balance tanks, or both. In one embodiment, the concentrations of components within the second evaporation concentrate bypass mixture and the second evaporation concentrate line mixture are about equal, or alternatively are exactly equal.

Following the separation step 26, the method 100A further includes subjecting at least a portion of the second evaporation concentrate line mixture to a fractionation chromatography step 28 to form a first fractionation portion and a second fractionation portion, wherein the first fractionation portion includes the galacto-oligosaccharide. In one embodiment, the entirety of the second evaporation concentrate line mixture is subjected to the fractionation chromatography step 28. The fractionation chromatography step 28 may involve the use of one or more of the following pieces of equipment, including but not limited to a simulated moving bed (SMB) chromatography device.

Following the fractionation chromatography step 28, the method 100A may include an optional second nanofiltration step 29 whereby at least a portion of the second fractionation portion (not containing the galacto-oligosaccharide) is subjected to nanofiltration to form a second nanofiltration retentate and a second nanofiltration permeate. In one embodiment, the entirety of the second fractionation portion is subjected to the optional second nanofiltration step 29. The optional second nanofiltration step 29 involves subjecting the second fractionation portion to one or more filtration sieves configured to separate out solids larger than the average pore size of the one or more filtration sieves. In one embodiment, the one or more filtration sieves used in the optional second nanofiltration step 29 have an average pore size greater than or equal to about 150 Daltons, less than or equal to about 300 Daltons, or alternatively between about 150 and 300 Daltons.

In embodiments wherein the second nanofiltration retentate is formed, it may be further processed, stored for later processing, or removed from further processing in the method 100A. In one embodiment, at least a portion of the second nanofiltration retentate is recombined with the first evaporation concentrate and subjected to the pH adjustment step 14 as described above. In one embodiment, the entirety of the second nanofiltration retentate is recombined with the first evaporation concentrate and subjected to the pH adjustment step 14 as described above. In another embodiment, at least a portion of the second nanofiltration retentate is stored or otherwise removed from further processing in the method 100A. In a further embodiment thereof, the entirety of the second nanofiltration retentate is stored or otherwise removed from further processing in the method 100A.

In embodiments of the invention including the optional second nanofiltration step, an optional second reverse osmosis step 31 may be included. When it is included, at least a portion of the second nanofiltration permeate is subjected to the second reverse osmosis step 31 to form a second reverse osmosis retentate and a second reverse osmosis permeate. In one embodiment, the entirety of the second nanofiltration permeate is subjected to the second reverse osmosis step 31.

At least a portion of the second reverse osmosis retentate may be removed from further processing in the method 100A, collected for further processing, collected to produce a product not described herein, or a combination thereof. In one embodiment, the entirety of the second reverse osmosis retentate is removed from further processing in the method 100A. In one embodiment, the entirety of the second reverse osmosis retentate is collected for further processing and/or to produce another product not described herein.

At least a portion of the second reverse osmosis permeate may be recycled for further processing at the optional second nanofiltration step 29 as described above, recycled for further processing at the optional condensate cooling step 13 as described above, or a combination thereof. In one embodiment, the entirety of the second reverse osmosis permeate is recycled for further processing at the optional second nanofiltration step 29. In one embodiment, the entirety of the second reverse osmosis permeate is recycled for further processing at the optional condensate cooling step 13.

Following the fractionation chromatography step 28, the method 100A further includes a mixing step 32 for mixing at least a portion of the first fractionation portion and at least a portion of the second evaporation concentrate bypass mixture to form a commodity mixture. In one embodiment, the entirety of the first fractionation portion is subjected to the mixing step 32. In one embodiment, the entirety of the second evaporation concentrate bypass mixture is subjected to the mixing step 32. In one embodiment, the entireties of both the first fractionation portion and the second evaporation concentrate bypass mixture are subjected to the mixing step 32. The resulting mixture from the mixing step 32 may proceed to be stored in storage tank (not shown) or a buffer tank (not shown) prior to further processing of the commodity mixture. In embodiments of the invention that operate at steady state, the commodity mixture may not need to be stored in a storage tank or a buffer tank prior to further processing, and may continue on to be subjected to a polishing ion exchange chromatography step 44 without intermediate storage.

Following the mixing step 32, at least a portion of the commodity mixture is subjected to a polishing ion exchange chromatography step 44 to remove one or more components that contribute to undesirable colors in the commodity mixture to form a polished commodity mixture. In one embodiment, the entirety of the commodity mixture is subjected to the polishing ion exchange chromatography step 44. In one embodiment, the one or more components that contribute to undesirable colors in the commodity mixture includes one or more components remaining in the commodity mixture originating from the clarified whey and not removed in prior steps, one or more components generated in one or more methods steps between the first ion exchange step 10 and the second ion exchange chromatography step 22, one or more components generated in one or more methods steps between the second ion exchange chromatography step 22 and the polishing ion exchange step 44, or a combination thereof. In one embodiment, the one or more components contributing to undesirable colors in the commodity mixture are mineral concentrations and/or ion concentrations. In a further embodiment, the polishing ion exchange chromatography step 44 is configured to reduce one or more mineral concentrations and/or ion concentrations, wherein the one or more mineral concentrations or ion concentrations reduced is selected from a list consisting of calcium, magnesium, phosphorus, potassium, sodium, and combinations thereof.

Following the polishing ion exchange chromatography step 44, at least a portion of the polished commodity mixture is subjected to a fourth evaporation step 46 to produce a fourth evaporation concentrate and a fourth evaporation condensate, wherein the fourth evaporation condensate comprises a dairy-derived galacto-oligosaccharide product. With respect to the method 100A, there is no third evaporation step 30 (discussed further below for methods 100B and 100C). In one embodiment, the entirety of the polished commodity mixture is subjected to the fourth evaporation step 46. Following the fourth evaporation step 46, a portion of or the entirety of the fourth evaporation condensate may optionally be subjected to the optional condensate cooling step 13 for further processing as described above

In one embodiment, the fourth evaporation concentrate defines the commodity grade dairy-derived galacto-oligosaccharide product without further processing. In one such embodiment, the fourth evaporation concentrate may be sent to an optional final storage tank (not shown) until it is ready for filling into packaging. In another embodiment, the fourth evaporation concentrate is further processed in an optional drying step 47 to produce a commodity grade, dried, dairy-derived, galacto-oligosaccharide product. Drying the dairy-derived galacto-oligosaccharide product may result in a powdered product. The commodity grade, dried, dairy-derived, galacto-oligosaccharide product may be stored in an optional final storage tank until it is ready for filling into packaging. In one embodiment, only a portion of the fourth evaporation concentrate is subjected to the optional drying step 47 such that both a commodity grade, dairy-derived, galacto-oligosaccharide product and a commodity grade, dried, dairy-derived, galacto-oligosaccharide product are produced.

With reference now to FIG. 2B which shows method 100B for producing the premium grade product, there is one key difference between the method 100B and the method 100A. Specifically, after the fractionation chromatography step 28 and before the mixing step 32, the method 100B further includes subjecting at least a portion the first fractionation portion to a third evaporation step 30 in order to form a third evaporation concentrate and a third evaporation condensate, followed by subjecting at least a portion of the third evaporation concentrate to the mixing step 32 along with the concentrate bypass mixture to define a premium mixture which is thereafter processed in the same way outlined above for the commodity mixture to produce premium grade products where method 100A teaches producing commodity grade products. In one embodiment of the method 100B, the entirety of the first fractionation portion is subjected to the third evaporation step 30. In one embodiment of the entirety of the third evaporation concentrate is subjected to the mixing step 32. Optionally, a portion of or the entirety of the third evaporation condensate may be subjected to the optional condensate cooling step 13 and further processed as outlined above. In other embodiments, an alternative means of removing water from the system may be used instead of evaporation in the third evaporation step 30 such as, by way of example and not limitation, an RO process.

With respect to further processing of the premium mixture in the method 100B as set forth above in the method 100A, it should be understood that subjecting a portion or the entirety of the premium mixture to the polishing ion exchange chromatography step 44 forms a polished premium mixture instead of a polished commodity mixture. It should be further understood that subjecting a portion or the entirety of the polished premium mixture to the fourth evaporation step 46 described above to form a fourth evaporation concentrate and a fourth evaporation condensate instead forms a fourth evaporation concentrate that defines a premium grade, dairy-derived, galacto-oligosaccharide product in method 100B. In other embodiments, an alternative means of removing water from the system may be used instead of evaporation in the fourth evaporation step 46 such as, by way of example and not limitation, an RO process. Moreover, a portion of or the entirety of the fourth evaporation condensate may still optionally be subjected to the optional condensate cooling step 13 for further processing as outlined for the method 100A above. Furthermore, it should be understood that subjecting the premium grade, dairy-derived, galacto-oligosaccharide product to the optional drying step 47 as described above instead forms a premium grade, dried, dairy-derived, galacto-oligosaccharide product.

Referring now to method 100C as shown in FIG. 2C, there are several additional steps when compared to the method 100B for processing the premium mixture following the mixing step 32 and prior to the polishing ion exchange chromatography step 44, ultimately resulting in super premium grade, dairy-derived, galacto-oligosaccharide products and super premium grade, dried, dairy-derived, galacto-oligosaccharide product.

Following the mixing step 32, at least a portion of the premium mixture is subjected to a cooling step 34 to form a cooled premium mixture. In one embodiment, the entirety of the premium mixture is subjected to the cooling step 34. The cooling step 34 involves reducing or maintaining the temperature of the premium mixture at a temperature at which a second reaction step 36 (discussed further below) may occur. In one embodiment, the cooling step 34 decreases the temperature of the premium mixture to a cooled temperature less than or equal to about 15° C., alternatively greater than or equal to about 9° C. and less than or equal to about 11° C., or alternatively about 10° C.

In a preferred embodiment, the cooled premium mixture has a pH between about 5 and about 6 when it is subjected to the second reaction step 36. Prior to the second reaction step 36, an optional second pH adjustment step (not shown) may be included in the method 100C to adjust the pH of the cooled premium mixture by adding a pH adjuster thereto prior to the second reaction step 36. In one embodiment, the optional pH adjustment step involves measuring the pH of the cooled premium mixture prior to adding the pH adjuster in order to determine whether adding a pH adjuster is desirable. The pH adjuster can either increase or decrease the pH of the cooled premium mixture as desired by adding either an acid to a cooled premium mixture having a pH greater than about 6 or, alternatively, adding a base to a cooled premium mixture having a pH less than about 5. Examples, but non-limiting, pH adjusters may be any pH adjuster selected from the group consisting of potassium hydroxide, sodium hydroxide, dipotassium phosphate, phosphoric acid, hydrochloric acid, lactic acid, and combinations thereof.

Following the cooling step 34 and/or the optional second pH adjustment step (not shown), at least a portion of the cooled premium mixture is subjected to a second reaction step 36 to form an enzyme premium mixture. In one embodiment, the entirety of the cooled premium mixture is subjected to the second reaction step 36. In one embodiment, the second reaction step 36 involves mixing the cooled premium mixture and the at least one enzyme in an enzyme reaction to form the enzyme premium mixture, which includes a greater amount of galacto-oligosaccharide when compared to the premium mixture. The at least one enzyme may comprise the same at least one enzyme or enzyme product discussed above for the first reaction step 16. In embodiments of the second reaction step 36 involving an enzyme reaction, the enzymes added during the enzyme reaction may include, but are not limited to one or more beta-lactase (also known as beta-galactosidase), which may come from a variety of sources. For example, the enzymes added during the reaction step 36 may come from Bifidobacterium bifidum, Aspergillus niger, Kluyveromyces lactis, or combinations thereof. In one embodiment, the enzymes added during the second reaction step 36 include beta-galactosidase (lactase) from Bifidobacterium bifidum at 1.5% of lactose content by mass. Alternatively, or in addition, the enzymes added during the second reaction step 36 may include beta-galactosidase from Aspergillus niger at 1.5% of lactose content by mass or Aspergillus niger at 0.75% lactose content by mass and Kluyveromyces lactis at 0.75% lactose content by mass. In one embodiment, the amount of the at least one enzyme may be greater than or equal to 0.25% lactose content by mass and less than or equal to 3% lactose content by mass, or alternatively greater than or equal to 0.25% lactose content by mass and less than or equal to 2% lactose content by mass, alternatively greater than or equal to 0.25% lactose content by mass and less than or equal to 1% lactose content by mass, alternatively greater than or equal to 1% lactose content by mass and less than or equal to 3% lactose content by mass, or alternatively greater than or equal to 1% lactose content by mass and less than or equal to 2% lactose content by mass.

In one embodiment of the second reaction step 36, a specific combination of enzymes is used including a lactose oxidase and/or a catalase in addition to the beta-galactosidase. In one such embodiment, the lactose oxidase is included in an amount greater than or equal to about 1% by mass and less than or equal to about 3% by mass. In one such embodiment, the catalase is included in an amount greater than or equal to about 0.75% by mass and less than or equal to 1% by mass. The catalase, when used, may come from the enzyme product Catazyme (produced by CHR Hansen Inc., version 6, including 1-5% catalase). The lactose oxidase, when used, may come from the enzyme product LactoYIELD (produced by CHR Hansen Inc., version 8). In one embodiment, both Catazyme and LactoYIELD are used together.

Additionally or alternatively, glucose oxidase may be included in addition to the beta-galactosidase to reduce the amount of glucose in the final product, which may increase the purity thereof. In one such embodiment, the amount of glucose oxidase is greater than or equal to about 1% by mass and less than or equal to about 3% by mass. The glucose oxidase, when used, may come from the enzyme product (supplied by DSM Food Specialties B.V., including 1-10% glucose oxidase).

In some embodiments, additional reagents are included, comprising compressed oxygen, compressed air, or both. In some embodiments, one or more buffering agent is included, wherein the one or more buffering agent may be selected from the list consisting of sodium hydroxide, potassium hydroxide, dipotassium phosphate, or a combination thereof.

When the reaction step 36 is completed, at least a portion of the enzyme premium mixture is subjected to an optional second enzyme deactivation step 38 to form a deactivated premium mixture. In one embodiment, the completion of the second reaction step 36 may require that no residual lactose is present in the enzyme premium mixture, or alternatively less than or equal to 0.01% lactose. In one embodiment, the entirety of the enzyme premium mixture is subjected to the optional second enzyme deactivation step 38. In one embodiment, the second enzyme deactivation step 38 comprises increasing the temperature of the enzyme premium mixture to a second threshold temperature. Without being bound by theory, such embodiments of this second enzyme deactivation step 38 work because when the at least one enzyme is subjected to a temperature above the second threshold temperature, the at least one enzyme is inactive in enzyme premium mixture is rendered inactive. Accordingly, the second threshold temperature may be any temperature, above which, the one or more enzymes used in the second reaction step are rendered inactive. In one embodiment, the second threshold temperature is 80° C. In one embodiment, the second enzyme deactivation step 38 is not included. In such embodiments, the non-deactivated at least one enzyme may be recycled for additional use via the optional enzyme recovery step 21 as described above.

Following the second enzyme deactivation step 38, at least a portion of the deactivated premium mixture is subjected to a third ultrafiltration step 40 to form a third ultrafiltration retentate and a third ultrafiltration permeate. Alternatively, in embodiments not including the optional enzyme deactivation step 38, at least a portion of the enzyme premium mixture is subjected to a third ultrafiltration step 40 to form a third ultrafiltration retentate and a third ultrafiltration permeate. In one embodiment, the entirety of the deactivated premium mixture or the enzyme premium mixture is subjected to the third ultrafiltration step 40. The third ultrafiltration step 40 involves subjecting the deactivated premium mixture or the enzyme premium mixture through one or more filtration sieves. In one embodiment, the one or more filtration sieves have an average pore size greater than or equal to about 5000 Daltons, alternatively less than or equal to about 10,000 Daltons, or alternatively between about 5,000 Daltons and about 10,000 Daltons. Excess water may optionally be used to increase the flow of the deactivated premium mixture through the one or more filtration sieves. In one such embodiment, the addition of water enables the third ultrafiltration step 40 to implement a diafiltration process. In one embodiment, the third ultrafiltration step 40 is configured to separate the one or more enzymes, which are relatively large compared to other components included in the deactivated premium mixture, from the other components included in the deactivated premium mixture. Optionally, at least a portion of the third ultrafiltration retentate, which contains the still active enzymes separated in this way, may be subjected to an optional enzyme recovery step 21 configured to recover the one or more enzymes for reuse in steps of the methods 100A, 100B, or 100C wherein enzymes are added such as, for example, the first reaction step 16, the second reaction step 36, or a combination thereof. Alternatively, a portion of or the entirety of the third ultrafiltration retentate may be stored or collected for other used not described herein. The third ultrafiltration permeate is sent for further processing.

Following the third ultrafiltration step 40, a portion of or the entirety of the third ultrafiltration permeate may be stored in an optional tank (not shown) prior to a third ion exchange chromatography step 42 (discussed further below). The optional tank may be used as a buffer tank, a storage tank, or both. In some embodiments, the optional tank may not be used or needed, especially in a steady-state manufacturing process in which storage of intermediate compositions is not needed.

Following the third ultrafiltration step 40, at least a portion of the third ultrafiltration permeate is subjected to a third ion exchange chromatography step 42, which further demineralizes and/or deionizes the third ultrafiltration permeate, to form a super-premium mixture by separating out ions of different charges included in the third ultrafiltration permeate. In one embodiment, the entirety of the third ultrafiltration permeate is subjected to the third ion exchange chromatography step 42. In one embodiment, the super-premium mixture includes fewer minerals, ions, and/or impurities that may have formed during the second reaction step 36 than the third ultrafiltration permeate. In one embodiment, the third ion exchange chromatography step 42 is configured to remove minerals and/or ions remaining in the third ultrafiltration permeate that originated from earlier steps in the process such as the clarified whey, minerals and/or ions formed prior to the formation of the first mineral-scarce mixture, minerals and/or ions formed between the first mineral scarce mixture and the second mineral scarce mixture, minerals and/or ions formed between the second mineral scarce mixture and the third ultrafiltration retentate, and combinations thereof. The third ion exchange chromatography step 42 also may remove components that contribute to undesirable colors to form the third ultrafiltration permeate. In some embodiments, one or more ion concentrations and/or mineral concentrations in the third ultrafiltration permeate is reduced during the third ion exchange chromatography step 42. For example, the one or more ion concentrations and/or mineral concentrations reduced in this way may be an ion or mineral selected from a group consisting of calcium, magnesium, phosphorus, potassium, sodium, and combinations thereof.

Following the third ion exchange chromatography step 42, a portion of or the entirety of the super-premium mixture is subjected to the polishing ion exchange chromatography step 44 and is further processed in the same way as described above for the commodity mixture in the description of method 100A above. With respect to further processing of the super-premium mixture in the method 100C as set forth above in the method 100A, it should be understood that subjecting a portion or the entirety of the super-premium mixture to the polishing ion exchange chromatography step 44 forms a polished, super-premium mixture instead of a polished commodity mixture. It should be further understood that subjecting a portion or the entirety of the polished, super-premium mixture to the fourth evaporation step described above to form a fourth evaporation concentrate and a fourth evaporation condensate instead forms a fourth evaporation concentrate that defines a super-premium grade, dairy-derived, galacto-oligosaccharide product in method 100C. Moreover, a portion of or the entirety of the fourth evaporation condensate may still optionally be subjected to the optional condensate cooling step 13 for further processing as outlined for the method 100A above. Furthermore, it should be understood that subjecting the super-premium grade, dairy-derived, galacto-oligosaccharide product to the optional drying step 47 as described above instead forms a super-premium grade, dried, dairy-derived, galacto-oligosaccharide product.

The methods 100A, 100B, and 100C are particularly well suited for producing products with high concentrations of galacto-oligosaccharide on a dry matter basis. This is particularly valuable for the production of natural products including galacto-oligosaccharide, especially in embodiments wherein the galacto-oligosachharides are formed according to methods 100A, 100B, and 100C using clarified whey derived from a dairy product or process. Additionally, by removing elements which do not improve the yields of this process (e.g., the first ultra filtration retentate, minerals, etc.), these removed elements can be repurposed for use in other products while maintaining a high output of the desired galacto-oligosaccharide.

While the present invention has been illustrated by a description of various embodiments and while these embodiments have been described in considerable detail, it is not the intention of the applicant to restrict or in any way limit the scope of the appended claims to such detail. By way of example, the methods and products discussed herein additionally or alternatively may be used to produce a dairy-derived galactooligosaccharide product or describe a dairy-derived galactooligosaccharide product itself. Additional advantages and modifications will readily appear to those skilled in the art. Thus, the invention in its broader aspects is therefore not limited to the specific details, representative apparatus, method, product, or illustrative example shown and described. Accordingly, departures may be made from such details without departing from the spirit or scope of applicant's general inventive concept.

What is claimed is:

1. A method for making a dairy-derived galacto-oligosaccharide product, the method comprising:
subjecting a pasteurized whey mixture having an original concentration of lactose to a first ultrafiltration step to form a first ultrafiltration retentate and a first ultrafiltration permeate;
subjecting the first ultrafiltration permeate to a first separation step to form a first separation permeate and a first separation retentate, wherein the first separation step consists of either a nanofiltration separation or a reverse osmosis separation;
subjecting the first separation retentate to a first evaporation step to form a first evaporation concentrate and a first evaporation condensate;
adjusting a pH of the first evaporation concentrate to a value greater than or equal to 3 and less than or equal to 9 to form a pH-adjusted first evaporation concentrate;
reacting, to form a galacto-oligosaccharide, at least one enzyme with the pH-adjusted first evaporation concentrate by adding the at least one enzyme to the pH-adjusted first evaporation concentrate to form a first reaction product comprising the galacto-oligosaccharide;
subjecting the first reaction product to a second ultrafiltration step to form a second ultrafiltration retentate and a second ultrafiltration permeate, the second ultrafiltration permeate being poorer in a protein concentration than the second ultrafiltration retentate;
subjecting the second ultrafiltration permeate to a second ion exchange chromatography step comprising reducing one or more mineral concentrations in the second ultrafiltration permeate to form a second mineral-scarce mixture;
subjecting the second mineral-scarce mixture to a second evaporation step to form a second evaporation concentrate and a second evaporation condensate;
separating a portion of the second evaporation concentrate to form a second evaporation concentrate bypass mixture and a second evaporation concentrate line mixture;
subjecting the second evaporation concentrate line mixture to a fractionation chromatography step to form a first fractionation portion and a second fractionation portion, wherein the first fractionation portion comprises the galacto-oligosaccharide;
mixing the first fractionation portion and the second evaporation concentrate bypass mixture to form a commodity mixture;
subjecting the commodity mixture to a polishing ion exchange chromatography step to form a polished commodity mixture; and
subjecting the polished commodity mixture to a third evaporation step to form a third evaporation condensate and a third evaporation concentrate, the third evaporation concentrate comprising the dairy-derived galacto-oligosaccharide product, the dairy-derived galacto-oligosaccharide product comprising lactose in a concentration less than the original concentration of lactose.

2. The method of claim 1, further comprising:
prior to subjecting the pasteurized whey mixture to the first ultrafiltration step, pasteurizing a clarified whey mixture comprising a dairy-derived acid whey and the original concentration of lactose, to form the pasteurized whey mixture.

3. The method of claim 2, wherein pasteurizing the clarified whey mixture comprises subjecting the clarified whey mixture to a high-temperature, short-time pasteurization process, wherein the high-temperature, short-time pasteurization process is conducted at a temperature greater than or equal to 161° F. and for a duration greater than or equal to 15 seconds.

4. The method of claim 1, wherein the first ultrafiltration step comprises filtering the pasteurized whey mixture through an ultrafilter having an average pore size of between 5,000 Daltons and 10,000 Daltons.

5. The method of claim 1, wherein the first separation step consists of a nanofiltration separation, and wherein the nanofiltration separation comprises filtering the first ultrafiltration permeate through a nanofilter having an average pore size of between 150 Daltons and 300 Daltons.

6. The method of claim 1 further comprising, prior to the first evaporation step, subjecting the first separation retentate to a first electrodialysis step comprising reducing one or more mineral concentrations in the first separation retentate.

7. The method of claim 6, wherein the one or more mineral concentrations reduced in the first electrodialysis step comprise a mineral selected from a group consisting of calcium, magnesium, phosphorus, potassium, sodium, and combinations thereof.

8. The method of claim 1 further comprising, prior to the first evaporation step, subjecting the first separation retentate to a first ion exchange chromatography step comprising reducing one or more mineral concentrations in the first separation retentate.

9. The method of claim 8, wherein the one or more mineral concentrations reduced in the first ion exchange chromatography step comprise a mineral selected from a group consisting of calcium, magnesium, phosphorus, potassium, sodium, and combinations thereof.

10. The method of claim 1, wherein adjusting the pH of the first evaporation concentrate comprises adjusting the pH to a value in a pH range selected from the group consisting of greater than or equal to 3 and less than or equal to 7, alternatively greater than or equal to 5 and less than or equal to 9, or alternatively greater than or equal to 5 and less than or equal to 6.

11. The method of claim 1, wherein adjusting the pH of the first evaporation concentrate comprises adding a pH adjuster selected from the group consisting of potassium hydroxide, sodium hydroxide, dipotassium phosphate, phosphoric acid, hydrochloric acid, lactic acid, and combinations thereof to the first evaporation concentrate.

12. The method of claim 1, wherein the at least one enzyme added to the pH-adjusted first evaporation concentrate comprises one or more beta-lactase.

13. The method of claim 1, further comprising deactivating the at least one enzyme by heating the first reaction product to a first threshold temperature, wherein the at least one enzyme is inactive in the first reaction product.

14. The method of claim 13, wherein the first threshold temperature is greater than or equal to 80 degrees Celsius.

15. The method of claim 1, wherein the second ultrafiltration step comprises filtering the deactivated evaporation concentrate through an ultrafilter having an average pore size of between 5,000 Daltons and 10,000 Daltons.

16. The method of claim 1, wherein the method does not comprise deactivating the at least one enzyme by heating the first reaction product to a first threshold temperature, and wherein the enzymes collected in the second ultrafiltration retentate are recycled for use in the first reaction step.

17. The method of claim 1, wherein the dairy-derived galacto-oligosaccharide product comprises a proximate composition comprising between 60% DMB and 75% DMB the galacto-oligosaccharide, less than or equal to 20% DMB lactose, less than or equal to 10% DMB glucose, and less than or equal to 10% DMB galactose.

18. The method of claim 1, further comprising subjecting the dairy-derived galacto-oligosaccharide product to a drying step.

19. A method for making a dairy-derived galacto-oligosaccharide product, the method comprising:
subjecting a pasteurized whey mixture having an original concentration of lactose to a first ultrafiltration step to form a first ultrafiltration retentate and a first ultrafiltration permeate;
subjecting the first ultrafiltration permeate to a first separation step to form a first separation permeate and a first separation retentate, wherein the first separation step consists of either a first nanofiltration process or a first reverse osmosis process;
subjecting the first separation retentate to a first evaporation step to form a first evaporation concentrate and a first evaporation condensate;
adjusting a pH of the first evaporation concentrate to a value greater than or equal to 3 and less than or equal to 9 to form a pH-adjusted first evaporation concentrate;
reacting, to form a galacto-oligosaccharide, at least one enzyme with the first evaporation concentrate by adding the at least one enzyme to the first evaporation concentrate to form a first reaction product comprising the galacto-oligosaccharide;
subjecting the first reaction product to a second ultrafiltration step to form a second ultrafiltration retentate and a second ultrafiltration permeate, the second ultrafiltration permeate being poorer in a protein concentration than the second ultrafiltration retentate;
subjecting the second ultrafiltration permeate to a second ion exchange chromatography step comprising reducing one or more mineral concentrations in the second ultrafiltration permeate to form a second mineral-reduced mixture;
subjecting the second mineral-reduced mixture to a second evaporation step to form a second evaporation concentrate and a second evaporation condensate;
separating a portion of the second evaporation concentrate to form a second evaporation concentrate bypass mixture and a second evaporation concentrate line mixture;
subjecting the second evaporation concentrate line mixture to a fractionation chromatography step to form a first fractionation portion and a second fractionation portion, wherein the first fractionation portion comprises the galacto-oligosaccharide;
subjecting the first fractionation portion to a third evaporation step to form a third evaporation concentrate and a third evaporation condensate;
mixing the third evaporation concentrate and the second evaporation concentrate bypass mixture to form a premium mixture;
subjecting the premium mixture to a polishing ion exchange chromatography step to form a polished premium mixture; and
subjecting the polished premium mixture to a fourth evaporation step to form a fourth evaporation condensate and a fourth evaporation concentrate, the fourth evaporation concentrate comprising the dairy-derived galacto-oligosaccharide product, the dairy-derived galacto-oligosaccharide product comprising lactose in a concentration less than the original concentration of lactose.

20. The method of claim 19, wherein the dairy-derived galacto-oligosaccharide product comprises a proximate composition comprising less than or equal to 85% DMB galacto-oligosaccharide, less than or equal to 5% DMB lactose, less than or equal to 2% DMB glucose, and less than or equal to 2% DMB galactose.

21. The method of claim 19, further comprising:
prior to subjecting the pasteurized whey mixture to the first ultrafiltration step, pasteurizing a clarified whey mixture comprising a dairy-derived acid whey and the original concentration of lactose, to form the pasteurized whey mixture.

22. The method of claim 19 further comprising, prior to the first evaporation step, subjecting the first separation retentate to a first electrodialysis step comprising reducing one or more mineral concentrations in the first separation retentate.

23. The method of claim 19 further comprising, prior to the first evaporation step, subjecting the first separation retentate to a first ion exchange chromatography step comprising reducing one or more mineral concentrations in the first separation retentate.

24. The method of claim 19, further comprising deactivating the at least one enzyme by heating the first reaction product to a first threshold temperature, wherein the at least one enzyme is inactive in the first reaction product.

25. The method of claim 19, wherein the method does not comprise deactivating the at least one enzyme by heating the first reaction product to a first threshold temperature, and wherein the enzymes collected in the second ultrafiltration retentate are recycled for use in the first reaction step.

26. The method of claim 19, further comprising subjecting the dairy-derived galacto-oligosaccharide product to a drying step.

27. A method for making a dairy-derived galacto-oligosaccharide product, the method comprising:
subjecting a pasteurized whey mixture having an original concentration of lactose to a first ultrafiltration step to form a first ultrafiltration retentate and a first ultrafiltration permeate;
subjecting the first ultrafiltration permeate to a first separation step to form a first separation permeate and a first separation retentate, wherein the first separation step consists of either a nanofiltration separation or a reverse osmosis separation;
subjecting the first separation retentate to a first evaporation step to form a first evaporation concentrate and a first evaporation condensate;
adjusting a pH of the first evaporation concentrate to a value greater than or equal to 3 and less than or equal to 9 to form a pH-adjusted first evaporation concentrate;
reacting, to form a galacto-oligosaccharide, a first at least one enzyme with the first evaporation concentrate by adding the first at least one enzyme to the first evaporation concentrate to form a first reaction product comprising the galacto-oligosaccharide;
subjecting the first reaction product to a second ultrafiltration step to form a second ultrafiltration retentate and a second ultrafiltration permeate, the second ultrafiltration permeate being poorer in a protein concentration than the second ultrafiltration retentate;
subjecting the second ultrafiltration permeate to a second ion exchange chromatography step comprising reducing one or more mineral concentrations in the second ultrafiltration permeate to form a second mineral-reduced mixture;
subjecting the second mineral-reduced mixture to a second evaporation step to form a second evaporation concentrate and a second evaporation condensate;
separating a portion of the second evaporation concentrate to form a second evaporation concentrate bypass mixture and a second evaporation concentrate line mixture;
subjecting the second evaporation concentrate line mixture to a fractionation chromatography step to form a first fractionation portion and a second fractionation portion, wherein the first fractionation portion comprises the galacto-oligosaccharide;
subjecting the first fractionation portion to a third evaporation step to form a third evaporation concentrate and a third evaporation condensate;
mixing the third evaporation concentrate and the second evaporation concentrate bypass mixture to form a premium mixture;
cooling the premium mixture to form a cooled premium mixture;
reacting, to form additional galacto-oligosaccharide, a second at least one enzyme with the cooled premium mixture to form an enzyme premium mixture;
subjecting the enzyme premium mixture to a third ultrafiltration step to form a third ultrafiltration permeate and a third ultrafiltration retentate;
subjecting the third ultrafiltration permeate to a third ion exchange chromatography step to form a super-premium mixture;
subjecting the super-premium mixture to a polishing ion exchange chromatography step to form a polished super-premium mixture; and
subjecting the polished super-premium mixture to a fourth evaporation step to form a fourth evaporation condensate and a fourth evaporation concentrate, the fourth evaporation concentrate comprising the dairy-derived galacto-oligosaccharide product, the dairy-derived galacto-oligosaccharide product comprising lactose in a concentration less than the original concentration of lactose.

28. The method of claim 27, wherein the dairy-derived galacto-oligosaccharide product comprises a proximate composition comprising less than or equal to 90% DMB galacto-oligosaccharide, less than or equal to 0.01% DMB lactose, less than or equal to 0.01% DMB glucose, and less than or equal to 2% DMB galactose.

29. The method of claim 27, further comprising:
prior to subjecting the pasteurized whey mixture to the first ultrafiltration step, pasteurizing a clarified whey mixture comprising a dairy-derived acid whey and the original concentration of lactose, to form the pasteurized whey mixture.

30. The method of claim 27 further comprising, prior to the first evaporation step, subjecting the first separation retentate to a first electrodialysis step comprising reducing one or more mineral concentrations in the first separation retentate.

31. The method of claim 27 further comprising, prior to the first evaporation step, subjecting the first separation retentate to a first ion exchange chromatography step comprising reducing one or more mineral concentrations in the first separation retentate.

32. The method of claim 27, further comprising deactivating the first at least one enzyme by heating the first reaction product to a first threshold temperature, wherein the at least one enzyme is inactive in the first reaction product.

33. The method of claim 27, wherein the method does not comprise deactivating the first at least one enzyme by heating the first reaction product to a first threshold temperature, and wherein the enzymes collected in the second ultrafiltration retentate are recycled for use in the first reaction step, the second reaction step, or both.

34. The method of claim 27, further comprising deactivating the second at least one enzyme by heating the enzyme premium mixture to a first threshold temperature, wherein the second at least one enzyme is inactive in the enzyme premium mixture.

35. The method of claim 27, wherein the method does not comprise deactivating the second at least one enzyme by heating the enzyme premium mixture to a first threshold temperature, and wherein the enzymes collected in the third ultrafiltration retentate are recycled for use in the first reaction step, the second reaction step, or both.

36. The method of claim 27, further comprising subjecting the dairy-derived galacto-oligosaccharide product to a drying step.
